(19) United States
(12) Patent Application Publication
(10) Pub. No.: US 2026/0067560 A1
(43) Pub. Date: Mar. 5, 2026

(54) METHOD FOR ACQUIRING A SET OF IMAGES OF AN OBJECT IN THE MOUTH

(71) Applicant: DENTAL MONITORING, Paris (FR)

(72) Inventors: Thomas PELLISSARD, Maisons-Alfort (FR); Guillaume GHYSELINCK, CANTIN (FR); Xavier DOREMUS, MONTROUGE (FR)

(21) Appl. No.: 19/106,502

(22) PCT Filed: Aug. 29, 2023
(86) PCT No.: PCT/EP2023/073681

§ 371 Date: Feb. 25, 2025

(30) Foreign Application Priority Data
Aug. 31, 2022 (FR) FR2208706

(51) Int. Cl. H04N 23/60; A61B 1/00; A61B 1/24; A61B 90/00; A61C 7/00; A61C 9/00; G06T 19/00; H04M 1/04; H04N 23/63

(52) U.S. Cl. CPC H04N 23/64; A61B 1/0005; A61B 1/24; A61C 7/002; A61C 9/0053; G06T 19/006; H04M 1/04; H04N 23/632; H04N 23/633; A61B 2090/365; G06T 2210/41

(57) ABSTRACT

A method for acquiring a set of images covering a target belonging to an object in the mouth of a user, the method comprising the following steps: 1) presenting to the user, on a screen and using spatially augmented reality with respect to the object in the mouth observed by an image acquisition apparatus, a multidimensional symbol or a set of multidimensional symbols the shape and/or the position of each symbol being determined so as to indicate to the user at least one predetermined acquisition condition suitable for acquiring such an image; 2) for each symbol, acquiring, using the acquisition apparatus, such an image when said at least one predetermined acquisition condition associated with said symbol is met, preferably when all the predetermined acquisition conditions associated with said symbol are met.

### TECHNICAL FIELD

The present invention relates to a method for acquiring a set of images of an object in the mouth, and in particular a dental object, particularly a dental arch of a user. The invention also relates to a device for implementing such a method.

### PRIOR ART

It is conventional to acquire a set of images covering a target located in the oral cavity of a user, and in particular of the user's dental arches, in order to analyze his dental situation, in particular before establishing an orthodontic treatment. An image “covers” a target when it represents that target at least partially. The set of images “covers” the target when it contains images covering the target from different viewing directions, in particular to provide precise three-dimensional information on the target.

An acquisition in a dental office, or more generally from a dental care professional, can involve significant costs and stress for the user.

Alternatively, the user can acquire the dental images himself, for example using his mobile phone. To acquire an image, the user usually looks into a mirror, which can make it difficult to position the phone precisely. What's more, until the user has consulted the gallery wherein the images are stored, that person has no idea whether the images are of good quality, or whether they correctly cover the intended target. Even when consulting the gallery, he can't accurately assess whether the coverage rate of the target by the set of images acquired is sufficient. Finally, acquisition can be labor-intensive if the user is required to repeat it. Finding these difficulties frustrating, the user may end the acquisition operation prematurely.

There is a need to facilitate the acquisition of a set of images of the mouth covering a target, in particular all or part of the dental arches, while limiting the risk of incomplete or poor quality acquisition.

One aim of the invention is to meet this need.

### DISCLOSURE OF THE INVENTION

The invention proposes a method for acquiring a set of images covering, preferably with a coverage rate greater than or equal to a coverage threshold, a target belonging to an object in the mouth of a user, for example a set of images covering the incisors or teeth (target) of the dental arch (object in the mouth) of the user.

According to a first main aspect of the invention, the method comprises the following steps:
1) presenting to the user, on a screen and using spatially augmented reality with respect to the object in the mouth observed by an image acquisition apparatus, preferably one being handled by the user, a multidimensional symbol or a set of multidimensional symbols, the shape and/or the position of each symbol being determined so as to indicate to the user at least one predetermined acquisition condition suitable for acquiring such an image;
2) for each symbol, acquiring, using the acquisition apparatus, such an image when said at least one predetermined acquisition condition associated with said symbol is met, preferably when all the predetermined acquisition conditions associated with said symbol are met.

As will be seen in greater detail later in the description, the shape of a multi-dimensional symbol in the space of the real scene observed by the acquisition apparatus makes it possible, when the user looks at the screen, to inform and guide him towards one or more acquisition conditions associated with said symbol and suitable for acquiring a desired image. A symbol presented in augmented reality thus provides particularly effective guide information.

A method according to the first main aspect of the invention may also particularly comprise one or more of the following optional features:
in step 2), an image is acquired by the acquisition apparatus only if said at least one predetermined acquisition condition associated with at least one symbol is met, preferably only if all the predetermined acquisition conditions associated with said symbol are met;
at least one symbol defines
a symbol axis, preferably an axis of revolution, an acquisition condition being an angular deviation between the optical axis of the acquisition apparatus and said symbol axis of less than 20°, preferably less than 10°, preferably less than 5°, preferably substantially zero, that is a substantially perfect alignment of the optical axis of the acquisition apparatus with said symbol axis, and/or
a dimension which, on the representation of the symbol on the screen, is variable as a function of the distance between the acquisition apparatus and said symbol in augmented reality, said acquisition condition being a predetermined specific value for said dimension or the belonging of said dimension to a predetermined specific range of values, for example a said dimension less than 1 mm;
prior to step 1), the user is taught, for each symbol, said at least one predetermined acquisition condition associated with said symbol; for example, the user is told that, to acquire the set of images, he must, for each symbol, align the axis of the symbol as closely as possible with the optical axis of the acquisition apparatus, and/or try to view the symbol on the screen so that said dimension is as close as possible to said specific value;
said teaching is carried out by means of a tutorial, the tutorial preferably making it possible to progressively teach all the functionalities of the computer program executed to implement the method, the tutorial being upgradeable on the basis of updates;
in step 2), a said image is automatically acquired with the acquisition apparatus if predetermined acquisition conditions defining a position in space and/or an orientation of the acquisition apparatus about its optical axis and including said at least one acquisition condition indicated by the symbol, are met;
in step 2), if such an image is acquired with the acquisition apparatus,
the appearance of said symbol is modified or said symbol is made to disappear, and/or
an audible signal is emitted, and/or
a score displayed on the screen and relating to a rate of coverage of the target by the images already acquired and/or relating to the duration for the acquisition of the images already acquired and/or relating to the quality of the images already acquired and/or relating to the usefulness of the images already acquired is modified;
when the set of images has been acquired, the user is presented, on the screen, with a ranking determined as a function of said score;
the screen displays the symbols on preview images representing the real scene observed by the acquisition apparatus or on views of a model representing, like said preview images, at least said object in the mouth or said target;
the screen displays a reference mark in a fixed position on the screen, the reference mark preferably having a constant shape, such a symbol preferably having a shape complementary to the reference mark when a predetermined acquisition condition associated with said symbol is met;
the target comprises more than 2, preferably more than 5 and/or less than 32 teeth and/or the set of images comprises more than 2, more than 5, more than 10 and/or less than 1,000 images taken under different respective acquisition conditions and/or the set of symbols comprises more than 2, more than 5 and/or less than 100 symbols;
the symbols preferably each have an axis oriented towards the center of the mouth of the user;
the symbols are preferably distributed in one plane, preferably in two planes, preferably in three planes, preferably so as to obtain images with observation axes in the occlusal plane, in a plane inclined to the occlusal plane so as to obtain top views relative to the occlusal plane and in a plane inclined to the occlusal plane so as to obtain bottom views relative to the occlusal plane;
in the space of the object in the mouth for the augmented reality representation, the symbols are outside the mouth of the user, so that the acquired images are extraoral.

In one embodiment, the symbol is a portion of the surface of the target, and the preview image, or equivalent image, displays a sight for visualizing the angle of the acquisition apparatus. The sight, in the extension of the optical axis, is displayed on the surface of the target, in the same way as the sight appears on an object when it is aimed by a firearm projecting a laser beam onto the object, depending on the direction of fire.

To orientate the acquisition apparatus correctly, the user must therefore aim the sight at the symbols represented on the surface of the target.

In this embodiment, the symbol can be a point or a surface. If it is a surface, its contour deforms according to the shape and distance of the surface of the target onto which it is being projected. The symbol can advantageously be used to indicate a distance of the acquisition apparatus from the target and/or an orientation of the acquisition apparatus around its optical axis.

The shape of the sight is non-limiting.

In particular, the symbol can be a thumbnail, that is a small image, attached to a tooth, such as a star in a video game.

The set of symbols preferably defines acquisition conditions for the acquisition of
at least one image taken facing the user and/or at least one image taken to the right of the user and/or at least one image taken to the left of the user, preferably at least one image taken facing the user and at least one image taken to the right of the user and at least one image taken to the left of the user; and/or
at least one image taken facing the user, on the one hand, and at least one image taken from above the user and/or at least one image taken from below the user, on the other hand; preferably at least one image taken facing the user and at least one image taken from above the user and at least one image taken from below the user; and/or
at least one image taken with the mouth open and/or at least one image taken with the mouth closed.

The invention further relates to a device for implementing a method according to the first main aspect of the invention, comprising
an image acquisition apparatus, preferably in the form of a cell phone;
a computer, preferably integrated into the acquisition apparatus or in communication with the acquisition apparatus, having a computer program comprising program code instructions for
in step 1), arranging and presenting to the user one or more multidimensional symbols on a screen, preferably the screen of the acquisition apparatus, in spatially augmented reality of the object in the mouth, the shape and/or position of a symbol being determined so as to indicate to the user at least one predetermined acquisition condition suitable for acquiring such an image;
preferably, in step 2), authorizing the acquisition, with the acquisition apparatus, of such an image only if said at least one predetermined acquisition condition associated with a symbol is met, and/or commanding the acquisition apparatus to acquire such an image only if said at least one predetermined acquisition condition is met, and/or updating a coverage level of the target by the acquired images, and preferably comparing the coverage level with a coverage threshold, and preferably presenting on the screen information about the coverage level and/or about the difference between the coverage threshold and the coverage level.

The acquisition apparatus is preferably
a cell phone and said screen is integrated into the cell phone or
a device comprising a holder equipped with a camera and designed to be held against the user during image acquisition, in particular against the user's teeth and/or gums, the screen being integrated into the holder or at a distance from the holder.

According to a second main aspect of the invention, the method aims to rapidly cover the target with a coverage rate greater than or equal to a coverage threshold, and the method comprises the following steps:
a) acquiring at least one image, preferably by the user, using an image acquisition apparatus, preferably a cell phone;
b) updating the target coverage level as a function of said at least one image acquired in step a);
c) if the coverage level is below the coverage threshold,
determining guide information that guides one toward suitable acquisition conditions for acquiring, with the image acquisition apparatus, an additional image that increases the coverage level;
presenting the user with the guide information and, preferably, information about the coverage level and/or about the difference between the coverage threshold and the coverage level, that is information enabling the user to know the progress of the acquisition of said set of images, and,
modifying, by the user, the position and/or orientation of the acquisition apparatus as a function of the guide information, and repeating step a), preferably in real time, the acquired images preferably being extracted from a video which the user views on a screen of the acquisition apparatus.

As will be seen in greater detail later in the description, the determination of guide information as a function of a coverage rate updated in real time advantageously facilitates the acquisition of images of a mouth of the user, and in particular the acquisition of dental images. In particular, the user receives real-time guide information, which makes acquisition more efficient, especially when the guide information is chosen to guide one towards optimal acquisition conditions enabling the acquisition of an additional image that maximizes the increase in coverage level.

With guidance, there is no need for the user to visit a dental care professional, or for the user to be supervised by a dental care professional to acquire images. Images can advantageously be acquired under precise acquisition conditions, without any special training. In particular, they can be acquired by the user himself or by one of his relatives. Notably, the method facilitates the acquisition of images of a child's arches by a parent.

The presentation of information about the coverage level is also particularly advantageous, as it effectively dissuades the user from interrupting the acquisition before it is complete. This makes acquisition particularly pleasant, as the user knows at all times how far he still has to go before acquiring the entire set of images. Acquisition can even be fun.

A method according to the second main aspect of the invention may also particularly comprise one or more of the following optional features:
is displayed on a screen, preferably on a screen of the acquisition apparatus, preferably a cell phone:
a representation of the target,
a representation of the part of the target covered by one or more images acquired in step a) or earlier steps a), and
preferably, a representation of the object in the mouth when the object in the mouth includes parts of the mouth of the user other than the target;
the guide information and/or the information about the coverage level and/or about the difference between the coverage threshold and the coverage level is/are presented in augmented reality in a preview image displayed on a screen of the acquisition apparatus and representing the real scene observed by the acquisition apparatus and/or in an image equivalent to the preview image representing a theoretical scene representing, symbolically or realistically, all or some of the elements of said real scene, in the same arrangement as in said real scene;
the equivalent image preferably comprises a view of a model of at least part of said real scene, a model being a digital three-dimensional model;
the information about the coverage level and/or about the difference between the coverage threshold and the coverage level is presented in a spatial reference frame so as to constitute so-called guide information;
the information about the coverage level and/or about the difference between the coverage threshold and the coverage level is presented on a representation of the target,
applying an appearance, preferably a color or texture, to a region of said representation that differs depending on whether said area is covered by at least one acquired image, or
applying an appearance to a symbol representing a predetermined part of said target, for example a tooth, said appearance being different depending on whether said part is covered or not, preferably by making the symbol appear or disappear on the screen of the acquisition apparatus, or
by applying an appearance to a symbol indicating at least one acquisition condition, said appearance being different depending on whether an image has been acquired with said at least one acquisition condition or not;
the target comprises a set of teeth and/or soft tissues, the preview image or equivalent image is updated in real time, preferably on the user's cell phone screen, the covered teeth being specifically marked, by a symbol or by applying an appearance enabling them to be distinguished from uncovered teeth, a tooth being “covered” when its initial surface to be covered is covered;
a plurality of symbols are anchored, in augmented reality, in the real scene observed by the acquisition apparatus, so as to appear on the preview image or on an equivalent image, each symbol being able, for example, to be anchored on a respective tooth of the user or to be anchored so as to indicate to the user suitable acquisition conditions for the acquisition of the additional image;
the symbols are three-dimensional symbols according to the first main aspect of the invention;
in step a), the user targets such a symbol, preferably two-dimensional or three-dimensional, with the acquisition apparatus, and when the target symbol is reached, preferably when it is at least partially superimposed on a fixed reference mark displayed on the screen of the acquisition apparatus, at least one image is acquired, preferably automatically, that is without specific user intervention;
the symbols are anchored on adjacent or non-adjacent teeth, in augmented reality, on the preview image or on an equivalent image, e.g. the symbols can be anchored regularly along the dental arch, e.g. every 2 or 3 teeth;
the symbols are anchored and/or shaped so as to define, optionally in cooperation with such a reference mark, predetermined acquisition conditions, preferably a distance of the acquisition apparatus from the target and/or an orientation of the acquisition apparatus about its optical axis and/or an angle of said optical axis with respect to the target;
in step b), said at least one image acquired in step a) is analyzed in order to identify a representation of the target on said image, preferably by means of a neural network, then the corresponding region in the preview image is marked, preferably by coloring it with a color associated with said target;
in step a), before or after image acquisition, the quality of the acquired or previewed image is evaluated, that is displayed on the screen of the acquisition apparatus, respectively, preferably at least the sharpness and/or contrast and/or color balance of the image, and/or the distance of the acquisition apparatus from the target and/or the orientation of the acquisition apparatus about its optical axis and/or the angle of said optical axis with respect to the target, and the image is acquired and/or the coverage ratio is updated only if the quality exceeds a predetermined quality threshold;
in step c),
a plurality of suitable acquisition conditions are tested for the acquisition, with the image acquisition apparatus, of an additional image increasing the coverage level, then
the guide information is determined in such a way that it guides one towards the optimum acquisition conditions, that is those suitable for acquiring, with the image acquisition apparatus, the additional image which increases the coverage level the most;
in step b),
each acquired image is submitted
to at least one first neural network trained to detect the target representation in the image and/or an image processing algorithm adapted to detect the target representation in the image, and
to a second neural network trained to determine the position and orientation of the acquisition apparatus during image acquisition, and thus the direction of the optical axis of the acquisition apparatus, that is the camera of the acquisition apparatus, relative to the target, and the distance of the acquisition apparatus from the target, then
said target representation is projected onto a model of the target or object in the mouth, or “reference model”, along a projection direction oriented, with respect to said model, as the direction of the optical axis with respect to the target, said target representation being virtually (that is theoretically) positioned, with respect to the reference model, as the acquisition apparatus is with respect to the target, then
the coverage rate is determined as a function of the area of the reference model covered by said projection and by said projections produced in any previous step(s) b);
in step b),
each acquired image is submitted
a first neural network trained to detect the representation of the target, or noteworthy points of said target, in the image, and to a second neural network trained to recognize, from the representation of the target or of said noteworthy points in the image, a corresponding region of a reference image, then
the coverage rate is determined as a function of the area of the reference image covered by said corresponding region and by said corresponding regions determined in any previous step(s) b);
the acquisition apparatus is
a cell phone with a screen, preferably the user's own cell phone, or
a device comprising
a holder equipped with a camera and held against the user during the acquisition of the set of images, preferably allowing the mouth to be opened and closed, preferably introduced partially into the mouth of the user, preferably resting against the gums and/or teeth, and
a screen displaying the scene observed by the camera, said screen being integrated in the holder or at a distance from the holder;
in step a), the user visualizes, in real time, on a screen of said cell phone, the actual scene observed by the cell phone or a corresponding theoretical scene, an image preferably being acquired automatically when the cell phone observes the target under predetermined acquisition conditions, preferably under acquisition conditions determined in a step c) of the preceding cycle of steps a) to c);
in step c), the user is presented, preferably on the screen of the acquisition apparatus, with a counter or gauge, preferably in the form of a progress bar, providing information about the coverage level and/or about the difference between the coverage threshold and the coverage level, and/or
when the coverage threshold is reached, the user is presented, preferably on the screen of the acquisition apparatus, with a score calculated as a function of the time taken to reach the coverage threshold and/or the quality of the images acquired, and/or the usefulness of the images acquired, and/or the user is presented, preferably on the screen of the acquisition apparatus, with a ranking determined as a function of said score.

In a first main embodiment, the guide information comprises a set of symbols, positioned, in augmented reality, as a function of the respective images to be acquired. The set of images to be acquired comprises one image for each symbol. In step a), the user must point his cell phone at the symbols and selects them, as in a video game. For example, each symbol can be anchored, in augmented reality, on a respective tooth, the target being constituted by said teeth and/or soft tissue.

The symbols can be anchored according to desired acquisition conditions, for example on non-adjacent teeth, for example every two or three teeth.

A reference mark may be depicted on the screen of the user's cellphone. When the reference mark overlaps the symbol, the latter is selected; an image is then acquired, preferably automatically, and the symbol is marked, or disappears. The marking or disappearance of symbols provides information about the coverage rate. It also provides guide information, as the user can easily spot symbols not yet selected on the preview image displayed on the cell phone screen. He can arrange the cell phone accordingly. Once all symbols have been selected, the set of images covers all targeted teeth and/or soft tissues. The coverage level can be, for example, the ratio of the number of symbols reached to the initial number of symbols, that is before the start of acquisition.

In one variant of this first main embodiment, the symbols are anchored and/or shaped so as to define, in cooperation with the reference mark, predetermined acquisition conditions, preferably a distance of the acquisition apparatus from the target and/or an orientation of the acquisition apparatus about its optical axis and/or an angle of said optical axis with respect to the target.

In one embodiment, the optional reference mark is shaped to be superimposed on a plurality of symbols simultaneously, for example two or three symbols. For example, it comprises a plurality of elementary reference marks, such as circles, which must be simultaneously superimposed on a plurality of respective symbols. Advantageously, this superimposing corresponds to a predetermined angle and distance of the acquisition apparatus from the target. Preferably, the symbols of a said plurality of symbols have an appearance, for example a color, specific to the plurality of symbols. For example, the user first aims to place the three green symbols in the circles of the reference mark, then the three red symbols, and so on.

Likewise, to impose a predetermined distance of the acquisition apparatus from the target, the reference mark and a symbol can have compatible dimensions, so that when the user superimposes the reference mark and the symbol exactly, the acquisition apparatus and the target are separated by said distance.

The symbols of a plurality of symbols and the elementary reference marks are preferably different from one another, e.g. they have different numbers. For example, the user aims to place the three symbols numbered 1, 2 and 3 in the circles numbered 1, 2 and 3 on the reference mark, respectively. Advantageously, if the elementary reference marks are not aligned, this superposition can impose a predetermined orientation of the acquisition apparatus around its optical axis.

Equivalently, to impose an orientation, the reference mark and a symbol can have a non-revolutionary shape, for example a rectangular shape, preferably a shape without symmetry, so that the user can only superimpose, with the same orientation, the reference mark and the symbol if the acquisition apparatus is oriented in one or more predetermined orientations around the optical axis.

In particular, when symbols are used to impose an angle, distance or orientation on the acquisition apparatus, they are not necessarily associated with particular teeth, and in particular with teeth to be covered.

In a second main embodiment, a view of a three-dimensional model of the target, for example a set of teeth, is displayed on the cell phone screen. In one embodiment, the user can modify this view, that is change the point of observation of the model, using conventional model manipulation software. In a preferred embodiment, the view is modified according to the conditions of observation of the target by the cell phone, preferably following the principles of augmented reality. The template can then be displayed, possibly transparently, superimposed on the preview image, or replace the preview image. When a preview image covers the target, an image is acquired, preferably automatically. The area of the target shown on the acquired image is marked on the model, preferably colored, for example green. The area of the target still to be covered is shown differently, for example colored red. The user can thus immediately see the coverage rate, for example the ratio between the green area and the total red and green area. The unmarked area guides him towards acquisition conditions that will increase the coverage rate.

In a variant of the second main embodiment, the reference model view is replaced by a reference image, e.g. a photograph, e.g. a panoramic photo.

Preferably, a method according to the invention is implemented to acquire
at least one image taken facing the user and/or at least one image taken to the right of the user and/or at least one image taken to the left of the user, preferably at least one image taken facing the user and at least one image taken to the right of the user and at least one image taken to the left of the user; and/or
at least one image taken facing the user, on the one hand, and at least one image taken from above the user and/or at least one image taken from below the user, on the other hand; preferably at least one image taken facing the user and at least one image taken from above the user and at least one image taken from below the user; and/or
at least one image taken with the mouth open and/or at least one image taken with the mouth closed.

The invention further relates to a device for implementing a method according to the invention, comprising:
an image acquisition apparatus, a camera-equipped computer, in particular a cell phone or tablet, preferably a cell phone, or a camera-equipped mirror for acquiring the set of images, in particular for carrying out step a);
a computer, which may be the acquisition apparatus, preferably integrated in the cell phone or in communication with the cell phone, having a computer program comprising program code instructions for according to the first main aspect of the invention, presenting three-dimensional symbols that provide guide information in augmented reality, and/or
according to the second main aspect of the invention
updating the coverage level in step b),
comparing the coverage level with the coverage threshold, and
if the coverage level is less than the coverage threshold, determining the guide information and presenting the guide information and, preferably, information about the coverage level and/or about the difference between the coverage threshold and the coverage level to the user in step c);
preferably a screen for displaying guide information and, according to the second main aspect of the invention, information about the coverage level and/or about the difference between the coverage threshold and the coverage level, preferably a computer screen.

In a preferred embodiment, the computer program is executed by the image acquisition apparatus, whereby the computer program can be integrated into specialized software, in particular specialized software for cell phones or tablets.

The screen can be integrated into the image acquisition apparatus. Preferably, the screen is the screen of a cell phone or tablet.

Preferably, the screen and computer program are integrated into the image acquisition apparatus.

The user can then easily, without the intervention of a third party, and in particular without the intervention of a dental care professional, by means of a simple cell phone or tablet, acquire images of the target in good quality and covering the entire target together.

The device may further comprise communication means, in particular for sending the acquired image(s) and/or receiving a model or a reference image.

Of course, insofar as they are not technically incompatible, the necessary or optional features of the various main aspects of the invention can be combined.

### Definitions

A “user” is a person for whom a method according to the invention is implemented.

The term “dental care professional” refers to any person qualified to provide dental care, including orthodontists and dentists.

An “arch” or “dental arch” means all or part of a dental arch, preferably comprising at least 2, preferably at least 3, more preferably at least 4 teeth. According to the international convention of the FDI World Dental Federation, each tooth in a dental arch has a predetermined number.

“Soft tissues” are those parts of the mouth covered with skin, such as the gums, palate or tongue, as opposed to teeth or braces.

Soft tissue is extra-skeletal support tissue, such as adipose tissue, tendons, ligaments, fascia, skin, etc. (soft connective tissue) and muscle, vascular and nerve tissue (non-connective tissue).

A “retractor” is a device for rolling up the lips or, more generally, for moving the lips away from the teeth. It preferably comprises an upper and a lower flange, and/or a right and a left flange, extending around a retractor opening and intended to be inserted between the teeth and the lips. In the operating position, the user's lips rest on these edges, so that the teeth are visible through the retractor opening. A retractor thus makes it possible to observe the teeth without being obstructed by the lips. However, the teeth do not rest on the retractor, so that by turning the head relative to the retractor, the user can change the teeth that are visible through the retractor opening. The user can also change the spacing between their dental arches. In particular, a retractor does not press on the teeth to spread the two jaws apart, but rather on the lips. In one embodiment, a retractor is configured to elastically spread the upper and lower lips apart to expose the teeth visible through the retractor opening. In one embodiment, a retractor is configured so that the distance between the top edge and the bottom edge, and/or between the right edge and the left edge, is constant. Retractor are described, for example, in PCT/EP2015/074896, U.S. Pat. No. 6,923,761, or US 2004/0209225.

By “computer” we mean a computer processing unit, which includes a set of several machines with computer processing capabilities. In particular, this unit can be integrated into a cell phone, in particular the cell phone of the user, or be a PC-type computer or server, for example a server remote from the user, e.g. being the “cloud” or a computer located at a dental care professional's premises. The cell phone and the computer then have the means to communicate with each other.

Typically, a computer comprises a processor, a memory, a human-machine interface, typically comprising a screen, and a communication module via the Internet, WIFI, Bluetooth® or the telephone network. Software configured to implement a method of the invention is loaded into the computer's memory. The computer can also be connected to a printer.

The method according to the invention (other than the acquisition operation, carried out by the image acquisition apparatus, and the operation of moving the image acquisition apparatus, carried out by the user) is implemented by computer, preferably exclusively by computer.

A “real scene” is made up of a set of elements observed simultaneously by the acquisition apparatus. The view of the actual scene observed by the acquisition apparatus is conventionally displayed on a screen of the acquisition apparatus, in the form of a “preview image” which is continuously updated in real time, like a film.

The preview image can be replaced or supplemented by an equivalent image representing, symbolically or realistically, a theoretical scene representing all or some of the elements of the real scene, in the same arrangement as in the real scene, that is in such a way that the elements represented are arranged, relative to one another, as in the real scene. The equivalent image is a view of the theoretical scene under observation conditions identical to those used by the acquisition apparatus to observe the real scene and obtain the preview image. The contours of physical element representations on the equivalent image are therefore superimposable on the contours of said physical elements on the preview image. The equivalent image is chosen to match the preview image as closely as possible.

The preview image is preferably displayed on the screen of the acquisition apparatus, but in one embodiment, the equivalent image replaces the preview image on the screen. However, acquiring an image with the acquisition apparatus at a given time consists of recording the preview image as it is displayed, or as it would have been displayed when replaced by an equivalent image.

Augmented reality is a form of communication in which visual elements are added to an image to represent, realistically or symbolically, a scene. In particular, visual elements can be added to a preview image representing the actual scene observed by the acquisition apparatus, or to an equivalent image.

In a preferred embodiment, augmented reality is used, so that when an image representing the target is acquired in step a), a screen represents, in real time, realistically or symbolically, the target as observed by the image acquisition apparatus.

A symbol is “anchored”, in augmented reality, when it appears as fixed in the real or theoretical scene when the acquisition apparatus moves in relation to the real scene (and therefore modifies the image displayed by the acquisition apparatus).

A symbol that appears in augmented reality can be a point, be two-dimensional, that is extend in a plane in the space of the object in the mouth, or preferably be three-dimensional, that is extend, virtually, in all three dimensions of the space of the object in the mouth. How the two-or three-dimensional symbol is represented depends on the conditions under which it is virtually observed. For example, its size depends on the observation distance. Preferably, the symbol is not spherical, so that its representation also provides information on the angle and/or orientation of the acquisition apparatus around its optical axis. A symbol providing guide information is preferably displayed on the preview image or equivalent.

The term “model” means a three-dimensional digital model. A model is made up of a set of voxels.

A “tooth model” is a three-dimensional digital model of a tooth. A dental arch model can be cut to define tooth models for at least some, preferably all, of the teeth represented in the arch model. Tooth models are therefore models within the arch model.

An “image” refers to a two-dimensional image, such as a photograph or a video frame. An image is made up of pixels. A “video” is considered to be a collection of photos. The number of pixels in an image is preferably greater than 100, 1,000, 10,000 or 100,000 or 1,000,000 and/or fewer than 1,000,000,000.

An image represents a scene, realistically or otherwise.

In particular, an image can represent a deformed mask, resulting from the projection, preferably by the acquisition apparatus, of an original mask. The original mask can be, for example, a grid or a set of dots, typically evenly distributed. The projection can be in the form of visible or non-visible light, preferably infra-red light. The deformation of a part of the original mask, for example a dot, resulting from its projection carries information about the distance between the region of the scene onto which this part of the original mask has been projected and the image acquisition apparatus. It can also provide information on the orientation of said region in space. The images used by Apple's Face ID software are examples of such images, also known as “3D images”.

An image acquired according to the invention is preferably a photo, possibly extracted from a video, realistically representing the observed scene, that is as perceived by the human eye. It can represent a distorted mask, in particular superimposed on an image depicting the scene realistically. It may only represent a distorted mask. As the original mask is projected onto the real scene observed by the acquisition apparatus, the image representing the deformed mask is also considered an image equivalent to the preview image.

The “match” or “fit” between two objects, for example between two images of a dental arch, is a measure of the difference, or “distance”, between them. A “best fit” is achieved when this difference is minimal, in particular when the two images represent the same elements in essentially the same way, that is in such a way that the element representations on these two images are essentially superimposable in alignment.

The “acquisition conditions” of an image specify the position and/or orientation in space of an image acquisition apparatus of this image relative to the target, and preferably the calibration of this image acquisition apparatus (in particular aperture, exposure time, focal length and sensitivity). A symbol can indicate acquisition conditions suitable for a single position and orientation of the acquisition apparatus. A symbol can alternatively indicate acquisition conditions corresponding to a plurality of positions and/or orientations of the acquisition apparatus, to guide towards several potential images. For example, it can guide towards a predetermined observation axis without guiding towards a particular position along this axis, thus leaving the user free to acquire one or more images along this axis at various positions along it, as in the embodiment of FIG. 3 for example. It can also guide to a predetermined position in space, without guiding to a particular orientation of the acquisition apparatus around its optical axis, as in the embodiment shown in FIG. 5, for example.

The image acquisition apparatus comprises a camera for acquiring images, such as photos or a film. When reference is made to an observation of a scene by the acquisition apparatus, reference is made to an observation of the scene by the camera of the acquisition apparatus. Where reference is made to an optical axis of the acquisition apparatus, reference is made to the optical axis of the acquisition apparatus's camera, and so on.

An “angle” is an orientation of the optical axis of the acquisition apparatus relative to the target.

An angle between two straight lines is an angle formed between two planes perpendicular to these two straight lines, respectively.

Unless otherwise indicated, “including” or “comprising” or “having” should be interpreted in a non-restrictive manner.

### BRIEF DESCRIPTION OF THE FIGURES

Further features and advantages of the invention will become apparent from the following detailed description and from an examination of the appended drawing, wherein:

FIG. 1 schematically shows the steps of a cycle of a method according to the second main aspect of the invention;

FIG. 2 shows an example device according to the invention;

FIG. 3 shows an example of implementing a method according to the second main aspect of the invention, in the first main embodiment;

FIG. 4 shows an example of a method according to the first main aspect of the invention and according to the second main aspect of the invention, in the second main embodiment;

FIG. 5 shows another example of a method according to the first main aspect of the invention and according to the second main aspect of the invention, in the second main embodiment;

FIG. 6 shows a symbol from FIG. 5 as presented to the user when predetermined acquisition conditions associated with the symbol are met;

FIG. 7 shows another example of a method according to the first main aspect of the invention and according to the second main aspect of the invention, in the second main embodiment;

FIG. 8 shows a schematic representation of a three-dimensional symbol viewed along its axis;

FIG. 9 schematically shows the steps of a cycle of a method according to the second main aspect of the invention;

FIG. 10 shows two examples of images equivalent to a preview image.

### DETAILED DESCRIPTION

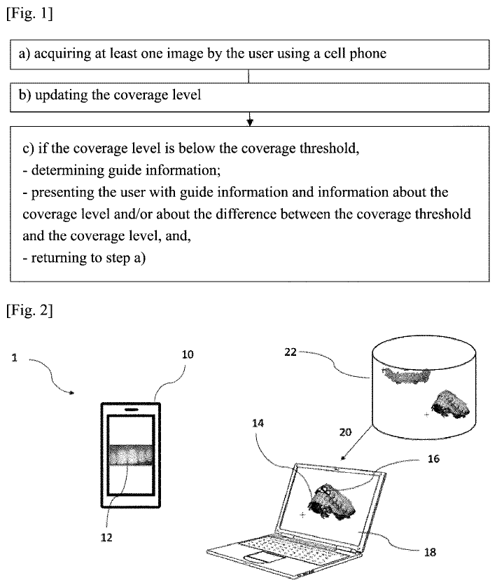

An acquisition method according to the invention, shown in FIG. 1, is for example implemented by means of the device 1 shown in FIG. 2.

The device 1 comprises
an image acquisition apparatus 10 provided with a screen 12 and into which a computer program is loaded, and
a computer 14 provided with a screen 18.

The computer 14 can be separate from the acquisition apparatus or, preferably, integrated into the acquisition apparatus.

The computer 14 may further comprise digital communication means for exchanging data 20, in particular with the image acquisition apparatus 10, or even with a database 22.

The database 22 can also be partially or fully integrated into the acquisition apparatus or computer. In particular, it can contain the acquired images, the reference model or reference image, the definition of the target and the object in the mouth, or even a final model generated from the acquired images. It can also contain information on the predetermined acquisition conditions associated with each symbol.

In a preferred embodiment of the invention, the image acquisition apparatus 10 is a cell phone or tablet. The screen 12 of the acquisition apparatus is configured to present guide information, and preferably information about the coverage level and/or about the difference between the coverage threshold and the coverage level. Alternatively, or additionally, this information can be presented on the computer screen 18.

The image acquisition apparatus can also be a mirror equipped with a camera.

The device 1 is used to implement a method according to the invention:

The target has a predetermined “initial coverage area”, that is an area that the method is intended to cover.

The “covered area” is that part of the initial surface to be covered which, at some point during the method, has already been covered, that is represented on at least one acquired image. The “area yet to be covered” is that part of the initial area to be covered which, at a given point during the method, has not been represented on any acquired image.

The area of a target can include elementary areas of several respective identifiable organs, for example a plurality of teeth. An organ can be described as “covered” or “yet to be covered”, depending on whether or not its entire elementary area is covered.

The object in the mouth may comprise, or consist of, the tongue and/or the palate and/or one or two gums and/or one or two dental arches, and/or one or more teeth. Preferably, the object in the mouth is a dental arch.

The object in the mouth can also be an orthodontic appliance, e.g. a multi-attachment appliance, vestibular or lingual, an orthodontic aligner, preferably invisible, an auxiliary, e.g. a cleat, a button or a screw, a functional training appliance, e.g. to modify tongue positioning or to treat sleep apnea.

A target can be the object in the mouth. It can also be a region of interest of the object in the mouth, in particular a region that has been identified as an at-risk region or a monitored region, for example as part of orthodontic treatment or periodontic follow-up, a region that has been poorly scanned, for example during a previous appointment with a dental care professional, or a region that has changed.

In a preferred embodiment, the object in the mouth is a dental arch and the target is one or preferably a plurality of teeth of said dental arch.

The target is identified before the method is implemented and the computer is informed. For example, the computer is told that it is to acquire a set of images covering teeth 10 to 14, or is provided with an image or model of a dental arch on which the target representation has been identified.

The image or model used to identify the target can be generic, that is it can be used by a plurality of users. They can be selected from a database, the database being accessible via digital communications. A generic model can be a typodont. Preferably, the generic model or generic image is chosen so that it represents a target with a shape close to the user's target, which improves the accuracy of the method. If the target belongs to a dental arch, the model can be generated by implementing a tooth model arrangement process, for example as described in European application no. 18 184486.

The model or image used to identify the target is preferably a model or image representing the user's target, acquired prior to implementing the method.

Each region of the target that is represented on an acquired image is said to be “covered” by that image. The union of the regions represented on at least one acquired image is the “area covered”.

The set of images is considered sufficient to cover a given target when the coverage level by the acquired images reaches a coverage threshold.

The coverage threshold thus defines, directly or indirectly, a percentage of the initial area to be covered which is considered sufficient for the acquisition to be terminated, that is for the acquisition to be considered completed.

A coverage threshold of 100% means, for example, that the entire surface of the target must be represented on at least one acquired image.

The coverage threshold can be set so that the set of images is sufficient to view the target from predetermined angles, in particular any angle.

The coverage threshold is preferably predetermined before the first step a).

The coverage threshold can be greater than 50%, 70%, 80%, 90% or 95% of the target area. Preferably, the coverage threshold is greater than 95%.

The coverage level is a measure of the progress of the acquisition relative to the coverage threshold. Before the first image is acquired, the coverage level is therefore zero. The level of progress increases progressively with each cycle of steps a) to c).

The coverage level can be, for example, the ratio of the area covered to the initial area to be covered. If 30% of the initial area to be covered is covered, the coverage level is thus 30%.

The coverage level can also be, for example, the ratio of the area covered to the area yet to be covered, made up of all the regions of the target that are not represented on any acquired image. If 30% of the initial area to be covered is covered, the coverage level is thus 30%/70%.

If the object in the mouth comprises a set of teeth, the coverage level can also be the ratio of the number of teeth covered to the number of teeth in said set. A tooth can be considered covered, for example, if at least 90% of the outer or occlusal or vestibular surface of the tooth is represented on the acquired images, better still if at least 95% of said tooth surface is represented on the acquired images, even better if all of said tooth surface is represented on the acquired images.

A tooth can alternatively be considered as covered when one or more noteworthy points of this tooth are represented on at least one acquired image.

In the embodiment described above, it has been assumed that the objective is to cover an area of the target.

By extension, in one embodiment, the aim is to acquire a set of images under respective acquisition conditions defined by respective symbols, preferably multidimensional. The set of symbols each defining acquisition conditions for one or more respective images can then be assimilated, at the start of implementation of a method according to the invention, to an “initial area to be covered”. The set of symbols defining, at an instant, acquisition conditions under which an image has already been acquired can be deemed a “covered area”, the number of these symbols defining a “coverage level”, the “coverage threshold” being able to be a minimum number for these symbols. Finally, the set of symbols defining acquisition conditions under which an image remains to be acquired can be deemed an “area yet to be covered”.

Also by extension, in one embodiment, the aim is to acquire a set of images partially representing the target, for example representing, for each tooth of the target, noteworthy points such as mesial-distal points, cusps, free edge, points along the neck, or the barycenter of a tooth face. At the start of a method according to the invention, the set of noteworthy points on the target can then be assimilated to an “initial surface to be covered”. The set of target noteworthy points already represented on an acquired image can be considered a “covered area”, the number of these points defining a “coverage level”, the “coverage threshold” being a minimum number for these points. Finally, the set of noteworthy points not yet represented on an already acquired image can be considered an “area yet to be covered”.

One aim of the method is to guide the user during image acquisition so that the set of images acquired comprises as few images as possible, that is that acquisition is efficient, but enough images so that the coverage threshold is reached.

The first main aspect of the invention is to guide acquisition by means of multidimensional symbols. The second main aspect of the invention is to guide acquisition by informing the user of the progress of the acquisition.

### First Main Aspect of the Invention

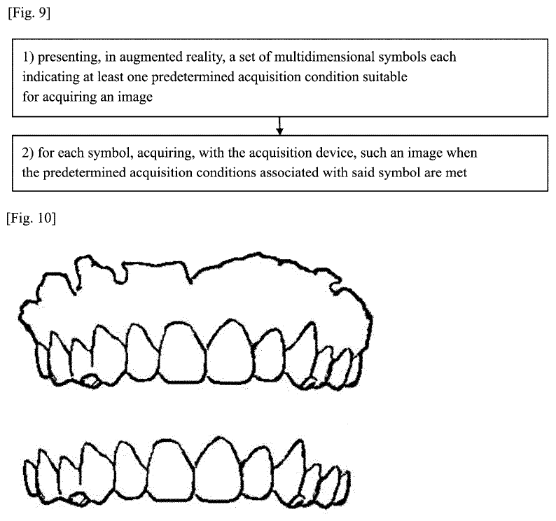

In step 1) of a method according to the first main aspect of the invention, shown in FIG. 9, multidimensional, that is two-dimensional or three-dimensional, preferably three-dimensional, symbols are arranged, that is “anchored”, and presented to the user, in augmented reality, in the space of the object in the mouth, the shape and/or position of a symbol being determined so as to indicate to the user all or part of acquisition conditions suitable for acquiring said image.

Of course, the user must be able to interpret a symbol. The information associated with a symbol can be explicit, for example when it takes the form of an arrow. No training is required. Alternatively, the user can be trained in order to provide him with the “rules of the game”, that is how he is expected to handle the symbols.

A two- or three-dimensional symbol that appears in augmented reality extends virtually, that is without having a physical existence, into the real scene observed by the acquisition apparatus, or into an equivalent virtual scene, in particular a model of at least part of the real scene. It extends in a plane or defines a volume, respectively.

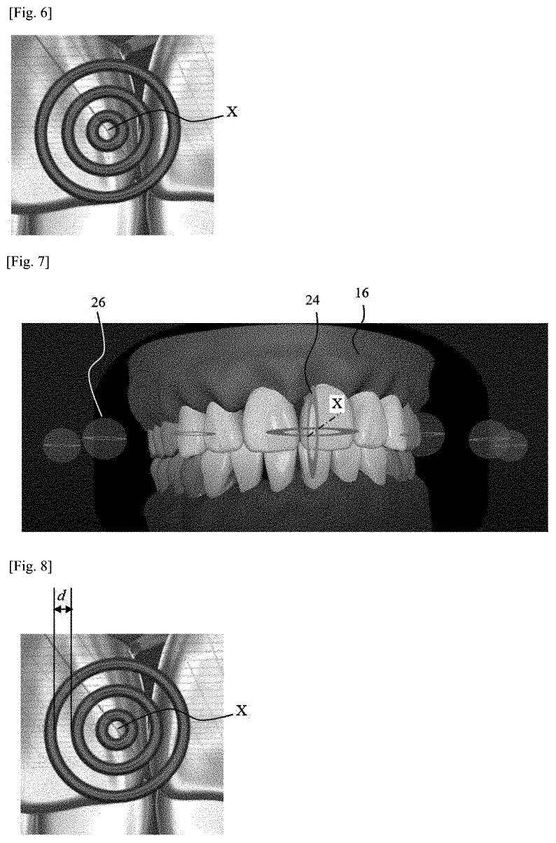

A representation in an equivalent virtual scene is advantageous, as shown in FIG. 7.

The screen displays the preview image representing the actual scene observed by the acquisition apparatus and/or an equivalent image, in particular a view of said model which corresponds to the observation of the actual scene by the acquisition apparatus. It also displays the symbols as if they had a real, physical existence and were present in the actual scene.

Each symbol is associated with one or more acquisition conditions for a respective image, in particular
a predetermined observation axis of the target or a predetermined range of observation axes, and/or
a predetermined distance between the target and the acquisition apparatus, or a range of said predetermined distances, and/or
a predetermined orientation of the acquisition apparatus around its optical axis, or a range of said predetermined orientations.

The computer knows the predetermined observation conditions associated with each symbol.

The number of symbols is adapted to the number of images in the desired set of images. It is preferably greater than 2, 5, 10, or 100 and/or smaller than 1,000.

A symbol, preferably each symbol, is preferably represented in the preview image or equivalent image, updated in real time as the user moves the acquisition apparatus.

A symbol, preferably each symbol, is preferably represented, in augmented reality, on a screen of the acquisition apparatus or in communication with the acquisition apparatus, preferably on a screen of a cell phone used to acquire images.

A symbol, preferably each symbol, is preferably anchored and/or shaped in such a way that the modification of its representation on said screen resulting from a movement of the acquisition apparatus, informs the user of the fact that said movement moves away from or towards predetermined acquisition conditions associated with said symbol.

The predetermined acquisition conditions define an axis of observation of the target by the acquisition apparatus, that is a predetermined axis of observation, and/or a predetermined distance of the acquisition apparatus from the target, preferably along the axis of observation, and/or a predetermined orientation of the acquisition apparatus about its optical axis, and said modification of the representation of the symbol on said screen preferably informs the user of the fact that said movement
reduces or increases the difference in angle between the optical axis of the acquisition apparatus and the predetermined observation axis, that is improves or degrades the angle of the acquisition apparatus; and/or
reduces or increases the difference between the distance between the acquisition apparatus and the target and the predetermined distance; and/or
reduces or increases the difference between the orientation of the acquisition apparatus about its optical axis and the predetermined orientation;
preferably reduces or increases each of the three above-mentioned differences.

The principles of augmented reality are well known. The determination of said angle and/or distance and/or difference in orientation poses no particular difficulty for the person skilled in the art.

Preferably, a multidimensional symbol, preferably each multidimensional symbol, has a shape defining a main direction, or “symbol axis”, identifiable by the user, for example an axis of revolution, and indicating a predetermined said axis of observation associated with said symbol.

A multidimensional symbol, preferably each multidimensional symbol, has, when the optical axis of the acquisition apparatus is coincident with said main direction, a dimension which, on the representation of the symbol on the screen, is variable as a function of the position of the acquisition apparatus along the optical axis, that is as a function of the distance between the acquisition apparatus and said symbol.

Said dimension can be evaluated by observation of the screen by the user, and the user knows a value of said dimension defining the position of the acquisition apparatus at the predetermined distance associated with said symbol.

For example, the multidimensional symbol takes the form of a superposition of rings, preferably of different diameters,
the predetermined orientation being obtained when the centers of the rings are aligned along the optical axis of the acquisition apparatus, that is said centers appear on the screen as merging, and/or
the predetermined distance being obtained when the centers of the rings are aligned along the optical axis and the spacing between said rings as they appear on the screen, preferably on the representation of the symbol in the preview image or equivalent image, has a predetermined value, for example when a first ring appears adjacent to a second ring, that is when the inner contour of the first ring is in contact with the outer contour of the second ring.

In step 2), an image is acquired when the predetermined acquisition condition(s) associated with a symbol is/are met.

In particular, acquisition can be carried out in step a) as described below. The image can be of the type described below for step a).

The image is then analyzed to determine the acquisition conditions.

In one embodiment, each acquired image is subjected to the following steps
a first neural network trained to detect the representation of the target, and/or noteworthy points of said target, in the image, and
a second neural network trained to recognize, from the representation of the target or of said noteworthy points in the image, said acquisition conditions, and in particular the angle and/or distance of the acquisition apparatus from the target.

The first neural network can be selected in particular from the Object Detection Networks, and in particular from the neural networks listed below, in the passage relating to step b2). For example, the neural network is trained by presenting it with 1,000 historical images:
as input, a historical image representing a historical target and/or noteworthy points, and
as an output, the representation of said historical target and/or noteworthy points on the historical image.

The neural network thus learns to recognize, in a new image, the representation of the target and/or noteworthy points.

The second neural network may in particular chosen from among networks specialized in image classification, called “CNN” (“Convolutional neural network”), e.g. AlexNet (2012), ZF Net (2013), VGG Net (2014), GoogleNet (2015), Microsoft ResNet (2015), Caffe: BAIR Reference CaffeNet, BAIR AlexNet, Torch: VGG_CNN_S, VGG_CNN_M, VGG_CNN_M_2048, VGG_CNN_M_1024, VGG_CNN_M_128, VGG_CNN_F, VGG ILSVRC-2014 16-layer, VGG ILSVRC-2014 19-layer, Network-in-Network (Imagenet & CIFAR-10), Google: Inception (V3, V4) For example, the neural network is trained by presenting it with, for more than 1,000 historical images: as input, a historical image representing a historical target and/or noteworthy points, and as output, the “historical”acquisition conditions of the historical image.

The neural network thus learns to define, for a new image, the conditions for its acquisition.

Determining the conditions for acquiring an image can also be achieved by searching for a view of a model of the user's arch that corresponds to the image, for example with an optimization operation, preferably a metaheuristic method, preferably evolutionary, preferably simulated annealing. An example of such a search is described, for example, in PCT/EP2015/074859, in European patent application no. 18 184477.0 or in WO2016/066651.

Preferably, when the symbol is observed along the predetermined observation axis, and/or when the acquisition apparatus is at said predetermined distance, and/or when the acquisition apparatus is oriented according to said predetermined orientation,
preferably, when the symbol is observed along the predetermined observation axis, and when the acquisition apparatus is at said predetermined distance, and when the acquisition apparatus is oriented according to said predetermined orientation,
an image is acquired, preferably automatically, that is without specific user intervention.

Preferably, a symbol changes appearance, for example color, or disappears when an image has been acquired under the acquisition conditions associated with said symbol.

### Second Main Aspect of the Invention

The method according to the second main aspect of the invention comprises a plurality of cycles of steps a) to c).

In step b), an image, preferably a photo, depicting an object in the mouth of a user, is acquired using an image acquisition apparatus. In one embodiment, a video is acquired with the image acquisition apparatus, and the acquired image is extracted from the video.

In one embodiment, an “original mask”, preferably a dot cloud, is projected onto the scene observed by the acquisition apparatus in step a), preferably by means of a projector integrated into the acquisition apparatus. The distorted mask resulting from the projection of the original mask then appears on the preview or equivalent image. In one embodiment, the projection is in infrared light so that the deformed mask is not visible to the naked eye. In one embodiment, the acquired image is the image representing the deformed mask. The acquisition apparatus preferably uses an infrared camera. However, the nature of the deformed mask is not limited.

The image is preferably acquired by the user himself. The user can acquire the image using a cell phone.

The acquired image is preferably “extraoral”, that is without the lens of the acquisition apparatus being inserted into the user's mouth.

The image acquisition apparatus can in particular be a cell phone, a tablet, a camera or a computer, the image acquisition apparatus preferably being a cell phone or a tablet, in particular so that the user can acquire images anywhere, and in particular outside a dental care professional's office, for example more than 1 km from a dental care professional's office.

In one embodiment, the user uses a cell phone and a holder to which the cell phone is removably attached, the holder being held against the user during the acquisition of at least some, preferably all of the images. In particular, the holder may be of the type described in PCT/EP2021/068702, EP17306361, PCT/EP2019/079565, PCT/EP2022/053847, FR2113577, FR2206750, or FR2206745.

In a preferred embodiment, the user uses a free cell phone, that is one whose position and orientation can be freely determined, and in particular not attached to a holder. In fact, the method described in the invention enables the user to be guided in taking images, so that guidance by means of a holder is not essential. Preferably, the image acquisition apparatus is not in contact with the user's mouth, either directly or via a holder for the image acquisition apparatus.

Beforehand, depending on the target, the computer or acquisition apparatus may ask the user to put an orthodontic appliance, such as an orthodontic aligner, cleat or archwire appliance, into or out of service. Alternatively, the user can be asked to move his lips away from his dental arches, preferably using a retractor, so as to better expose the target to the image acquisition apparatus, for example to fully expose at least one tooth, in particular the outer surface of an incisor and/or at least partially the outer surface of a molar. Alternatively, the user can be asked to open his mouth wide to acquire occlusal images showing the lingual and occlusal surfaces of the teeth and palate.

The number of images acquired in step a) is preferably less than 100, preferably less than 50, most preferably less than 10, so that the guide information can be updated quickly.

Before implementing the first step a) of a method according to the invention, the coverage level is zero.

In step b), the coverage level is updated to take into account the image(s) acquired in the immediately preceding step a), or “new images”.

In step b), the computer analyzes each new image, preferably according to the following steps:
b1) determining a potential contribution by the new image;
b2) determining the intersection between the potential contribution and the previous contribution from previously analyzed images;
b3) if the intersection is not empty, adding the potential contribution to the previous contribution.

In step b1), the computer determines the potential contribution of the new image. In particular, it determines whether the new image at least partially represents the target. If not, this new image cannot contribute and the computer moves on to analyze the next new image. If so, the computer determines the potential contribution of the new image, for example determining the contour of the target representation on the new image, or the number of the target tooth or teeth represented, at least partially, in the new image.

In step b2), the computer then compares the potential contribution to the set of contributions resulting from the analysis of previously analyzed images, or “previous contribution”.

For example, the potential contribution of the new image is the representation of the target in the new image. The computer evaluates the intersection between this potential contribution and the previous contribution, made up of the union of all the representations of the target on previously analyzed images.

If this intersection is empty, the new image cannot make a new contribution and the computer moves on to analyze the next new image. If this intersection is not empty, that is the new image represents a region of the target that was not in any of the previously analyzed images, the computer adds to the previous contribution a new contribution made up of said intersection.

In another example, the potential contribution of the new image is the number of one or more target teeth identified in the new image. The computer evaluates the intersection between this potential contribution and the previous contribution, made up of the set of tooth numbers of the target identified in the previously analyzed images. If this intersection is empty, the new image cannot make a further contribution and the computer moves on to analyze the next new image. If this intersection is not empty, the computer adds it to the previous contribution.

The potential contribution of a new image can be determined by any known means.

In one embodiment, this involves segmenting the new image so as to identify any total or partial representation of the target. Analysis can be performed using conventional segmentation methods.

In particular, the new image can be submitted to a neural network trained to detect the representation of the target on the new image, for example to determine the numbers of the teeth represented in the image, and/or the contours of said teeth, and/or of the mouth and/or of the lips, and/or of the tongue, as described for example in European patent application no. 18 184477.0.

Networks specialized in locating and detecting objects in an image are well known. The neural network can be selected in particular from Object Detection Networks, for example R-CNN (2013), SSD (Single Shot MultiBox Detector: Object Detection network), Faster R-CNN (Faster Region-based Convolutional Network method: Object Detection network), Faster R-CNN (2015), SSD (2015), RCF (Richer Convolutional Features for Edge Detection) (2017), SPP-Net, 2014, OverFeat (Sermanet et al.), 2013, GoogleNet (Szegedy et al.), 2015, VGGNet (Simonyan and Zisserman), 2014, R-CNN (Girshick et al.), 2014, Fast R-CNN (Girshick et al.), 2015, ResNet (He et al.), 2016, Faster R-CNN (Ren et al.), 2016, FPN (Lin et al.), 2016, YOLO (Redmon et al.), 2016, SSD (Liu et al.), 2016, ResNet v2 (He et al.), 2016, R-FCN (Dai et al.), 2016, ResNeXt (Lin et al.), 2017, DenseNet (Huang et al.), 2017, DPN (Chen et al.), 2017, YOL09000 (Redmon and Farhadi), 2017, Hourglass (Newell et al.), 2016, MobileNet (Howard et al.), 2017, DCN (Dai et al.), 2017, RetinaNet (Lin et al.), 2017, Mask R-CNN (He et al.), 2017, RefineDet (Zhang et al.), 2018, Cascade RCNN (Cai et al.), 2018, NASNet (Zoph et al.), 2019, CornerNet (Law and Deng), 2018, FSAF (Zhu et al.), 2019, SENet (Hu et al.), 2018, ExtremeNet (Zhou et al.), 2019, NAS-FPN (Ghiasi et al.), 2019, Detnas (Chen et al.), 2019, FCOS (Tian et al.), 2019, CenterNet (Duan et al.), 2019, EfficientNet (Tan and Le), 2019, or AlexNet (Krizhevsky et al.), 2012.

The above list is not exhaustive. For example, a neural network is trained by presenting it with 1,000 historical images:
as input, a historical image representing a historical target, and
as output, the representation of said historical target on the historical image.

The neural network thus learns to recognize, in a new image, the representation of the target.

When the intersection between the potential contribution and the previous contribution comprises a comparison of the representations of the target in the new image and in the previously analyzed images, these representations are preferably projected onto a common reference model in order to take into account different acquisition conditions, and in particular an orientation of the acquisition apparatus that varies according to the image under consideration.

The reference model preferably represents a reference object in the mouth, preferably similar or even identical to the object in the mouth of the user.

To project an image, the computer analyzes the image to determine its acquisition conditions, that is the actual acquisition conditions. In particular, it evaluates the distance between the acquisition apparatus and the object in the mouth of the user and the orientation of the acquisition apparatus in space, relative to the object in the mouth of the user, at the time of image acquisition.

Determination of the actual acquisition conditions can be carried out as described, for example, in European Patent Application No. 18 184477.0 or in WO2016/066651, or by subjecting the image to a neural network trained to determine the acquisition conditions of the image being submitted to it.

The actual acquisition conditions are then virtually reproduced in relation to the reference model, and the user's target representation in the image is projected onto the reference model.

The set of projected surfaces obtained from previous images can constitute the previous contribution. The projected surface obtained from the new image is the potential contribution.

Preferably, each image is submitted to a first detection neural network, identifying object in the mouths represented in the image, in particular, the tongue and/or tooth numbers, and/or gums and/or mouth and/or lips and/or noteworthy points of these organs, then each image is submitted to a second neural network trained to determine the acquisition conditions of the image it is submitted to. Preferably, the second neural network takes as input the object in the mouths detected by the first neural network, which improves the determination of acquisition conditions.

In one embodiment, the potential contribution of a new image is determined by comparing the image with a “reference” image, for example a photo or panoramic shot, preferably of at least one dental arch similar or identical to the user's dental arch.

The target is then generally not represented in the same way on the reference image and on the new image. Preferably, a neural network is trained to match the objects represented in the two images.

For example, it is trained by presenting to it:
as input, historical records each comprising a historical image representing a historical target under possible acquisition conditions with the acquisition apparatus, and an identification of the representation of the historical target in the historical image, and
as output, identification of said historical target on a historical reference image.

The neural network thus learns to recognize, in a reference image, the representation of the target that corresponds to the representation of the target in a new image submitted to it. The neural network thus learns to identify the potential contribution of the new image.

The method can be repeated several times, each time using a new reference image.

In step b3), the computer adds the potential contribution to the previous contribution if the intersection is not empty, and calculates the coverage level resulting from the incrementation of the previous contribution by the new contribution.

In a preferred embodiment, the computer evaluates the quality of the acquired image(s) and only adds the potential contribution in step b3) if the quality is above a predefined quality threshold. In particular, the quality can be an assessment of the image's sharpness and/or contrast and/or color balance, and/or the distance between the acquisition apparatus and the mouth of the user. Advantageously, only images of satisfactory quality are taken into account.

In one embodiment, the image acquisition apparatus, preferably in the form of a cell phone, is attached to a holder that is held in contact with the user during acquisition, as described above, for example a holder of the type described in PCT/EP2021/068702, EP17306361, PCT/EP2019/079565, PCT/EP2022/053847, FR2113577, FR2206750, or FR2206745. With such a holder, image quality (in particular brightness, the distance of the acquisition apparatus from the target, the angle of the acquisition apparatus relative to the target and the orientation of the acquisition apparatus around its optical axis) is advantageously well controlled, so that evaluation of the quality of the image or images acquired is optional.

Preferably, the holder is not bitten by the user, and, even more preferably, allows opening and closing (to a position wherein the arches are in contact with each other in the occlusal plane) of the arches.

Quality assessment is particularly advantageous in the absence of such a holder.

Depending on the result, the computer may decide to take several images, for example varying the focal length to acquire a first clear image of the incisor group, then a second clear image of the posterior group of teeth.

The acquisition of several images with different calibration conditions can be based on quality assessment, but can also be programmed to be routine at each acquisition stage.

In step c), the computer compares the coverage level with the coverage threshold. If the coverage level is greater than or equal to the coverage threshold, step c) is complete. Alternatively, the computer determines guide information to guide the user to position the image acquisition apparatus towards “future” acquisition conditions suitable for acquiring, in the next step a), an additional image increasing said coverage level.

The guide information is thus determined to inform the user of the areas of the target for which he still needs to acquire one or more images. It is presented to the user, guiding him to orient and/or position the image acquisition apparatus according to the future acquisition conditions to be adopted for the next step a).

In a preferred embodiment, the computer determines, for example by a random search or with an optimization algorithm, the future acquisition conditions for the next cycle, so that image acquisition under these future acquisition conditions maximizes the increase in coverage level.

The presentation of guide information can include visual, audible and/or haptic, in particular tactile, transmission of guide information.

For example, the guide information may comprise audio instructions telling the user to move the image acquisition apparatus towards or away from the teeth, to shift the image acquisition apparatus to the right or left, to rotate the image acquisition apparatus around the dental arch, to open or close the mouth, or to open or close the jaw.

A tactile transmission of guide information can be a vibration, for example, indicating to the user to stop a movement.

A haptic transmission of guide information can be a vibration, for example to indicate the successful passage of a reference mark over a surface of the target or symbol.

The presentation of the guide information can be adapted to the user.

Preferably, the presentation of guide information comprises several different types of transmissions stimulating several different senses, thus facilitating communication to the user.

Preferably, the guide information is presented on a screen, preferably on a screen of the image acquisition apparatus. The display of guide information on a screen may comprise
preferably a reference frame enabling the user to find his bearings when moving the acquisition apparatus in space, that is to evaluate how the acquisition apparatus is positioned relative to the object in the mouth, and in particular relative to the target, and
an indicator showing movement relative to this reference frame.

The reference frame preferably represents, at least partially, in a symbolic or realistic way (that is adapted so that the user recognizes the represented object), at least one object in the mouth as observed by the acquisition apparatus. It is determined according to the actual acquisition conditions of the acquisition apparatus.

The reference frame can be, for example, a preview image, that is the image observed by the acquisition apparatus, preferably the cell phone, in real time and displayed on the screen of the acquisition apparatus, and/or an equivalent image representing a part of the user, for example, the user's head or part of the head or the mouth of the user or dental arches (“equivalent” means that the image corresponds to an observation of the part of the patient that can be superimposed with the image observed by the acquisition apparatus, and in particular observed along the optical axis of the acquisition apparatus).

The equivalent image can be a line drawing, for example representing the outline of a part of the user.

The reference frame can represent a view of a user-specific model or a generic model, said model thus representing, precisely or more coarsely, a part of the user, preferably at least the target, preferably at least the object in the mouth.

A generic model is common to a plurality of individuals. In particular, a generic reference frame can be determined by statistical analysis of historical data representative of these individuals. A generic model can be, for example, a typodont model.

A user-specific model can be a model of all or part of the object in the mouth of the user, in particular the user's target. In particular, it can be a scan of the user's dental arches. It may also include or be a 3D model of a user's arch in a configuration specific to a treatment stage. In particular, it can comprise or be a 3D model of a user's arch in a configuration suitable for a stage of treatment with orthodontic aligners, and in particular a 3D model used for the design and manufacture of an orthodontic aligner. Such a 3D model can be generated at the start of orthodontic treatment, or during orthodontic treatment.

The equivalent image is preferably a view of a generic or specific model. Preferably, a texture is applied to the model to make it more realistic and make it easier for the user to identify with the model. The texture can be extracted from an image, for example an image acquired in step a), and then applied to a model, preferably selected from a database prior to step a).

The model for which a view is used as a reference can be, in particular, the reference model used as a projection medium for acquired images, as described above.

The equivalent image may be at least partly symbolic. It may comprise, for example, a set of geometric shapes representing the object in the mouth, for example a set of discs, each disc representing a tooth of a part of a dental arch, the object in the mouth being the dental arch.

FIG. 10 shows two examples of equivalent images, representing the view of a 3D model of the user's arch, with and without gums respectively. The view could also be, for example, a wireframe representation.

Particularly when the equivalent image is a view of a generic or specific model, the image acquisition apparatus can display the preview image, preferably in a “thumbnail” window, which facilitates spatial location for the user.

Displaying the reference frame on the screen is optional if the indicator provides an indication of the desired movement. For example, the indicator may be an arrow or a message recommending a particular movement. However, displaying the reference frame on the screen is preferred, as it considerably facilitates precise positioning of the acquisition apparatus.

The indicator is displayed, preferably together with the reference frame, on the screen, preferably on the screen of the image acquisition apparatus, preferably on a cell phone or tablet screen.

In a preferred embodiment, the reference frame comprises a representation of the object in the mouth and the indicator is a mark indicating a region of this representation not yet covered. The indicator can be, for example, a particular contour surrounding this area or, preferably, a particular color applied to this area, or a symbol superimposed on this area. A “special” contour or color is one that enables the user to distinguish said area from the rest of the representation of the object in the mouth.

This display helps to guide the user quickly and efficiently. This guidance system, which gives the user a great deal of freedom, is intuitive, so no prior training is required to be guided.

In particular, the indicator can be displayed transparently or highlighted on the representation of the object in the mouth.

The indicator is preferably displayed in augmented reality when the reference frame is a preview image or equivalent.

### Coverage Rate and Threshold

Preferably, in step c), the user is informed, preferably in real time, of the coverage level achieved, that is the progress of the acquisition, and preferably of the coverage threshold.

Preferably, coverage level information and/or coverage threshold information is/are presented on a screen, preferably on the user's cell phone screen. The information can take the form of a meter or a gauge, for example in the form of a progress bar.

In a preferred embodiment, the coverage level and/or coverage threshold is/are represented “graphically” on the screen, in particular in the form of line(s) and/or area(s) and/or symbols.

In one embodiment, the screen displays:
the initial area to be covered,
the area covered, and
preferably, a representation of elements of the target context, e.g. parts of the object in the mouth other than the target, that is parts of the object in the mouth which the method is not intended to cover, e.g. teeth adjacent to the teeth for which images are to be acquired.

For example, when the reference frame is a symbolic or preferably realistic representation of the object in the mouth or target, the initial area to be covered can be presented on the screen in a way that can be identified by the user, to inform him of the coverage threshold. In particular, it can be colored with a specific color, or more generally represented with a specific appearance, or delimited by a specific outline. The area thus represented with a specific appearance or encircled by this outline represents the coverage threshold.

Similarly, the area covered, that is for which at least one image has already been acquired, can be presented on the screen in a way that can be identified by the user, to inform him of the coverage level. In particular, it can be colored with a specific color, or more generally represented with a specific appearance, or delimited by a specific outline. The area(s) thus represented with a specific appearance or encircled by this outline represent the coverage level.

For example, a surface of the target may be displayed in green or red depending on whether acquisition of this surface has been completed or whether acquisition of this surface remains to be done. The area covered can be displayed transparently or highlighted.

In one embodiment, the coverage threshold is represented graphically as a set of symbols displayed in close proximity, preferably superimposed, on representations of a respective set of teeth. The representations of these teeth belong to, or even constitute, a reference frame. For example, the symbols can be presented in augmented reality on the cell phone's preview image or on a view of a dental arch model, preferably on a view of a model of the user's dental arches. The appearance of symbols relating to teeth for which the desired images have already been acquired (“covered teeth”) may differ from that of symbols relating to teeth for which not all the desired images have yet been acquired (“teeth yet to be covered”), enabling the coverage rate to be graphically seen.

In one embodiment, the tooth symbol disappears as soon as the tooth is covered. The user then sees the difference between the coverage threshold (all symbols initially displayed) and the coverage level (symbols that have disappeared).

The “graphic” display of coverage threshold and coverage level is particularly effective in ensuring that the user acquires all the images required.

Remarkably, the graphical representations of the coverage threshold and coverage level show the areas of the target still to be covered, that is for which the desired images have yet to be acquired. These graphical representations can be used as indicators to guide the user. For example, coloring the covered area with a color different from the area yet to be covered highlights the area yet to be covered, and thus guides the user.

The graphic, or “visual”, marking of the initial area to be covered, the area covered or the area still to be covered is not limited to the application of a color or texture or outline or the representation of particular symbols.

Preferably, the graphical representations of the coverage threshold and coverage level are displayed in augmented reality, preferably on the cell phone screen.

In one embodiment, a stopwatch is activated to measure the duration of image acquisition since the first step a). Displaying this time and the coverage level and/or the difference between the coverage threshold and the coverage level, is a motivating factor for the user.

In one embodiment, a score is calculated as a function of the time taken to reach the coverage threshold and/or the quality of the images acquired, and/or the usefulness of the images acquired, more generally a goal set for the user.

In one embodiment, the initial area to be covered comprises regions assigned a utility coefficient, and the score is determined as a function of the utility coefficients of the regions in the covered area. For example, in one embodiment, the initial surface to be covered may consist of a part that is essential to cover and a part that is optional to cover. The utility coefficient assigned to a pixel in the “essential” section can be 100, for example, and the utility coefficient assigned to a pixel in the “optional” section can be 10, for example. The score can, for example, be a function, or even the sum of the utility coefficients for all the pixels in the area covered.

The score can be compared with scores previously achieved by the user or by other users, to obtain a ranking of the set of images acquisition operation.

A ranking can be established for several patients, for example for all the patients of the same practitioner. An information message and/or a gift, such as a reward, can be sent to a patient according to his ranking order.

The stopwatch and/or score and/or ranking can be displayed on the acquisition apparatus screen.

This makes acquisition fun. In particular, acquisition can be presented as a video game, with the aim of reaching the coverage threshold as quickly as possible.

Guided by the guide information and motivated by the coverage level information, the user modifies the acquisition conditions, enabling a new step a) to be resumed, preferably immediately at the end of step c).

When the coverage level is presented to the user, he can immediately visualize the effect of moving the acquisition apparatus, in particular when the target becomes colored or when symbols associated with teeth change appearance or disappear as the image acquisition progresses. The guiding is highly intuitive.

In a preferred embodiment, the screen displays a realistic representation of the object in the mouth and the surface covered at that moment. The area covered is completed as the cycles progress, enabling the user to easily identify the area still to be acquired, and to position and orient the image acquisition apparatus accordingly.

The time interval between two successive cycles of steps a) to c) is preferably less than 5 minutes, 1 minute, 30 seconds or 1 second. Preferably, the user acquires the acquired images in real time, preferably by filming the object in the mouth, steps b) to c) being carried out immediately for each acquired image.

At the end of the cycles of steps a) to c), that is when the coverage level is greater than or equal to the coverage threshold, the acquired images can be transmitted to the user and/or preferably to a dental care professional.

Acquired images can be stored, for example in a database, preferably accessible to a dental care professional and/or the user. For example, the images acquired can be stored in a user's medical file.

The set of acquired images typically comprises more than 2, more than 5, more than 10, more than 50, more than 100 and/or less than 10,000 images.

Said set of acquired images can be used, in particular, to:
assess the user's dental situation, before, during or after orthodontic or dental treatment; and/or
generate or correct a model of the user's dental arch; and/or
design an orthodontic appliance, and/or
design and propose dental treatments, such as hygiene, periodontics or whitening.

In a particular embodiment, at the end of the cycles of steps a) to c), the method may involve analyzing the acquired images to generate a model of the target, referred to as the “final model”. The final model can be sent to the user, preferably a dental care professional. The final model can depict the target with a high degree of accuracy. The final model can be produced by the dental care professional's computer, to which the acquired images are transmitted, or by the image acquisition apparatus. The final model can be stored, for example in a database, preferably accessible to a dental care professional and/or the user. For example, the final model can be stored in the user's medical file.

### EXAMPLES

The aim of the acquisition is to acquire a set of images of a target consisting, for example, of all the user's teeth. The dental arches, which make up the object in the mouth, comprise the teeth and gums. The object in the mouth is modeled in the form of a computer-accessible reference model 16. The reference model 16 may come from a database and may be generic. The target is identified on the reference model. The initial area to be covered is therefore the target area in the reference model.

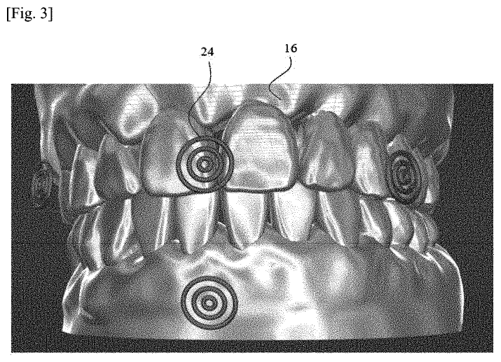

FIGS. 3, 5, 6 and 7 show examples of the first main aspect of the invention, wherein multidimensional symbols 24 are virtually arranged in space to guide the user to associated acquisition conditions.

In the example shown in FIG. 3, the symbols 24 are two-dimensional. They each take the form of three concentric, coplanar rings. They are represented on a view of a reference model representing arches, said view observing the model as the acquisition apparatus observes the user's dental arches. The view is preferably presented on the screen of a phone used for acquisition. The user's aim is to position the phone so as to see a target head-on, that is so that the rings appear circular. The optical axis then coincides with a predetermined observation axis suitable for image acquisition.

No reference mark is required for this guiding. However, a reference mark representing, for example, the circular contours of the rings when viewed from the front would improve the positioning accuracy of the acquisition apparatus.

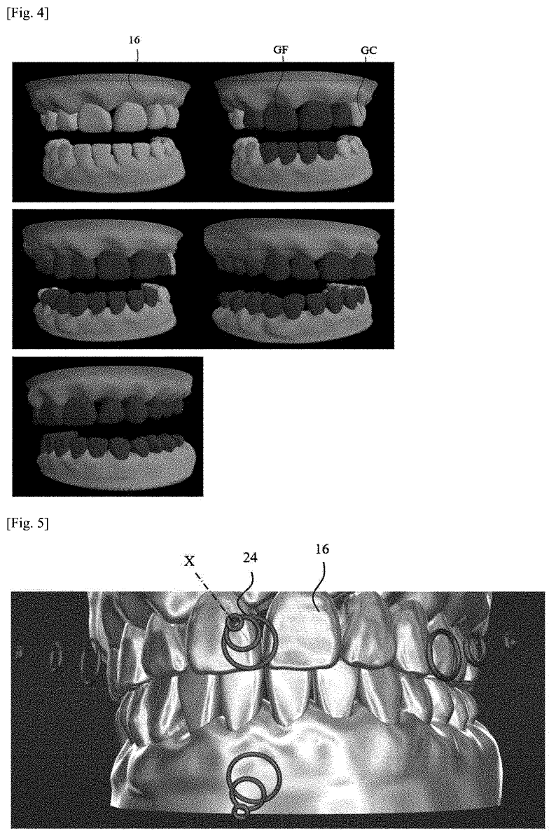

In the example shown in FIG. 5, the symbols are three-dimensional. They each take the form of three concentric, superimposed rings. They are represented on a view of a reference model representing arches, said view observing the model as the acquisition apparatus observes the user's dental arches. The view is preferably presented on the screen of a phone used for acquisition. The user's aim is to position the phone so as to see a target head-on, that is so that the rings appear circular and concentric, as in FIG. 6. The optical axis then coincides with a predetermined observation axis, namely the X axis of the rings, suitable for image acquisition.

In the example shown in FIG. 7, each symbol 24 consists of two concentric hoops extending in planes at 90°to each other. The two hoops are preferably of the same shape (same inside and outside diameters), and preferably enclosed in a sphere of color 26, but transparent to make them easier to locate. The intersection of the two planes defines a predetermined observation axis X associated with the symbol, identifiable by the user. This form of symbol makes it very easy to determine how to move the acquisition apparatus.

In the embodiment shown in FIG. 7, the first, horizontal hoop is used to determine whether the acquisition apparatus should be moved up or down, and the second, vertical hoop, in a plane passing through the center of the mouth, is used to determine whether the acquisition apparatus should be moved right or left.

Guiding with a three-dimensional symbol is particularly effective. No reference mark is required for precise positioning of the optical axis of the acquisition apparatus along the predetermined observation axis suitable for image acquisition.

A three-dimensional symbol is also used to guide to a predetermined distance along the predetermined observation axis. In the example shown in FIG. 8, the distance d between the rings is representative of the distance between the acquisition apparatus and the symbol, along the X axis of the symbol, that is along the predetermined observation axis. The predetermined distance may correspond, for example, to a view of the symbol wherein the rings touch (d=0). It's easy for the user to move the acquisition apparatus to this position.

When predetermined acquisition conditions associated with a symbol are reached, an image is acquired, preferably automatically, and preferably the appearance, e.g. color, of the symbol is modified, or the symbol disappears.

An example illustrating the second main aspect of the invention is now described:
In step a), an image is acquired using the acquisition apparatus.
In step b), the acquired image is analyzed to update the coverage level.

In particular, we look for the possible representation of the target on the acquired image, that is the existence of a potential contribution from the acquired image.

If this potential contribution exists, a search is made for the view of the reference model that shows maximum agreement with the acquired image, and from this the area of said view that corresponds to the target is deduced, and consequently the corresponding zone on the reference model. If part of the latter surface has not yet been registered as belonging to the covered area, it is added to the covered area, and marked on the reference model, preferably colored a first color specific to the covered area, e.g. green, the remainder of the area of the target on said view preferably a second color, e.g. red, and the area of the reference model not defining the target preferably a third different color, e.g. white.

The view of the reference model 16 equivalent to the preview image is projected onto the screen of the image acquisition apparatus 10, preferably a cell phone, in the direction of the optical axis.

The projected view of the reference model may
be superimposed almost exactly with the preview image displayed on the screen, with or without transparency, or
replace the preview image, as shown in FIG. 4.

It allows the user to see which areas are covered and which are yet to be acquired, thanks to their specific colors.

This presentation enables the user to quickly and easily identify the areas of teeth yet to be covered, and to easily orientate the acquisition apparatus accordingly. It also informs the user of the coverage level.

As images are acquired during the cycles of steps a) to c), the area covered increases.

The images in FIG. 4 each represent a view of a reference model representing two dental arches, preferably similar to those of the user. Each view corresponds to a preview image and is displayed in place of said preview image, preferably on the screen of a cell phone used for image acquisition. The reference model views are therefore “equivalent” to preview images. In other words, when the user turns the acquisition apparatus around his mouth, the displayed reference model view immediately adapts accordingly. Users can therefore easily assimilate the reference model to their dental arches. He can therefore easily position and orient the acquisition apparatus to acquire images of teeth not yet covered.

In the embodiment shown in FIG. 4, the target consists of all the teeth of both arches, and the object in the mouth consists of both arches (and therefore includes the gums as well as the teeth). Tooth color is specifically dark gray (GF) when tooth coverage is sufficient, and light gray (GS) otherwise.

As the user moves the acquisition apparatus in front of his teeth, the surface area covered increases, and therefore the number of teeth sufficiently covered, shown in dark grey, increases. The other teeth remain light gray.

The coverage level corresponds, for example, to the percentage of area covered relative to the initial area to be covered. The coverage threshold can be a percentage of the target area to be covered. For example, the threshold may be 90%, that is when more than 90% of the tooth surface belongs to the covered area, the coverage threshold is reached.

Symbols, possibly one-dimensional, that is in the form of a point, can symbolically represent the teeth to be covered (target). When, for example, at least 90% of a tooth's surface is acquired, better still when at least 95% of a tooth's surface is covered, even better still when the entire surface of a tooth is covered, the symbol symbolically representing this tooth is no longer shown on the screen. Alternatively, the symbol is displayed in color or highlighted.

As is now clear, a device and a method according to the invention advantageously make it possible to increase the user's autonomy and improve the quality and content of images acquired by a user with no particular knowledge in the dental field. They can also be used to produce a 3D model of a target belonging to or constituting an object in the mouth of the user, from a distance. Finally, they make it much easier to determine orthodontic treatment at a distance, as well as to follow up any orthodontic treatment, without the user having to make an appointment with a dental care professional.

Of course, the invention is not limited to the above-described and illustrated embodiments.

In particular, the cell phone can be replaced by a device comprising a holder equipped with a camera and held against the user during the acquisition of the set of images, and a screen displaying the scene observed by the camera, said screen being integrated into the holder or at a distance from the holder.

The shape of the symbols is not limiting. Symbols in 1, 2 or 3 dimensions can be presented simultaneously in augmented reality.

1. A method for acquiring a set of images covering a target belonging to an object in the mouth of a user, the method comprising the following steps:
1) presenting to the user, on a screen and using spatially augmented reality with respect to the object in the mouth observed by an image acquisition apparatus, a multidimensional symbol or a set of multidimensional symbols the shape and/or the position of each symbol being determined so as to indicate to the user at least one predetermined acquisition condition suitable for acquiring such an image; and,
2) for each symbol, acquiring, using the acquisition apparatus, such an image when said at least one predetermined acquisition condition associated with said symbol is met, preferably when all the predetermined acquisition conditions associated with said symbol are met.

2. The method according to claim 1, wherein, in step 2), an image is acquired with the acquisition apparatus only if a predetermined acquisition condition associated with at least one symbol is met.

3. The method according to claim 1, wherein at least one symbol defines
a symbol axis (X), preferably an axis of revolution, said acquisition condition being an angular deviation between the optical axis of the acquisition apparatus and said symbol axis of less than 20°%; and/or,
a dimension (d) which, on the representation of the symbol on the screen, is variable as a function of the distance between the acquisition apparatus and said symbol in augmented reality, said acquisition condition being a specific value for said dimension or the belonging of said dimension to a predetermined specific range of values.

4. The method according to claim 1, wherein, in step 2), a said image is automatically acquired with the acquisition apparatus if predetermined acquisition conditions defining a position in space and/or an orientation of the acquisition apparatus around its optical axis and including said at least one acquisition condition indicated by the symbol, are met.

5. The method according to claim 1, wherein, in step 2), such an image is acquired with the acquisition apparatus,
the appearance of said symbol is modified or said symbol is made to disappear, and/or
an audible signal is emitted; and/or,
a score displayed on the screen and relating to a rate of coverage of the target by the images already acquired and/or relating to the duration for the acquisition of the images already acquired and/or relating to the quality of the images already acquired and/or relating to the usefulness of the images already acquired is modified.

6. The method according to the claim 5, wherein, when the set of images has been acquired, the user is presented, on the screen, with a ranking determined as a function of said score.

7. The method according to a claim 1, wherein the screen displays the symbols on preview images representing the real scene observed by the acquisition apparatus or on views of a model representing, like said preview images, at least said object in the mouth or said target.

8. The method according to claim 1, wherein the screen displays a reference mark in a fixed position on the screen, such a symbol preferably having a shape complementary to the reference mark when a predetermined acquisition condition associated with said symbol is met.

9. The method according to claim 1, wherein the target comprises more than 5 teeth and/or an orthodontic appliance, and/or the set of symbols comprises more than 2 symbols.

10. The method according to claim 1, wherein the acquisition apparatus is manipulated by the user in steps 1) and 2).

11. The acquisition method according to claim 1, wherein the images are photos, preferably realistic, and/or represent a deformed mask, resulting from the projection, preferably by the acquisition apparatus, of an original mask, preferably in the form of a grid or a set of dots.

12. A device for carrying out a method according to claim 1, comprising
an image acquisition apparatus, preferably in the form of a cell phone;
a computer, preferably integrated into the acquisition apparatus or in communication with the acquisition apparatus, having a computer program comprising program code instructions for
in step 1), arranging and presenting to the user one or more multidimensional symbols on a screen preferably on the screen of the acquisition apparatus, in augmented reality within the space of the oral object, the shape and/or position of a symbol being determined to indicate to the user at least one predetermined acquisition condition suitable for acquiring such an image; and,
preferably, in step 2), authorizing the acquisition, with the acquisition apparatus, of such an image only if said at least one predetermined acquisition condition associated with a symbol is met, and/or commanding the acquisition apparatus to acquire such an image only if said at least one predetermined acquisition condition is met, and/or updating a coverage level of the target by the acquired images, and preferably comparing the coverage level with a coverage threshold, and preferably presenting on the screen an information about the coverage level and/or about the difference between the coverage threshold and the coverage level.

13. The device according to claim 12, wherein the acquisition apparatus is
a cell phone and said screen is integrated into the cell phone; or,
a device comprising a holder equipped with a camera and held against the user during image acquisition, the screen being integrated into the holder or at a distance from the holder.
